(19) United States
(12) Patent Application Publication
(10) Pub. No.: US 2026/0053759 A1
(43) Pub. Date: Feb. 26, 2026

(54) MESDOPETAM COMPOSITIONS

(71) Applicant: ALKEM LABORATORIES LIMITED, Mumbai (IN)

(72) Inventors: Akhilesh Sharma, Mumbai (IN); Anindita Chatterjee, Mumbai (IN); Ulhas Dhuppad, Nashik (IN)

(21) Appl. No.: 19/105,624

(22) PCT Filed: Aug. 22, 2023
(86) PCT No.: PCT/IN2023/050791

§ 371 Date: Feb. 21, 2025

(30) Foreign Application Priority Data
Aug. 24, 2022 (IN) 202221010032

(51) Int. Cl. A61K 31/145; A61K 9/00; A61K 9/16; A61K 9/20; A61K 31/4439; A61P 1/00

(52) U.S. Cl. CPC A61K 31/145; A61K 9/0053; A61K 9/1652; A61K 9/1682; A61K 9/2009; A61K 9/2013; A61K 9/2054; A61K 9/2095; A61K 31/4439; A61P 1/00

(57) ABSTRACT

The present invention relates to a composition comprising Mesdopetam alone or in combination with proton pump inhibitor which can be administered either separately i.e. by co-administration or in a fixed dose combination (FDC) in the treatment of gastrointestinal and related disorders. The present invention provides use of mesdopetam in the manufacture of a medicament for the treatment of gastrointestinal disorders in a subject in need thereof wherein the medicament is to be administered to the subject. The present invention also relates to a method of treating gastrointestinal diseases in a subject in need thereof comprising administering to the subject mesdopetam and pharmaceutical composition.

### FIELD OF THE INVENTION

The present invention relates to compositions of Mesdopetam alone or in combination with proton pump inhibitors (PPIs). In particular, but without restriction to the particular embodiments hereinafter described in accordance with the best mode of practice, the present invention relates to a composition comprising Mesdopetam alone or in combination with proton pump inhibitors (PPIs), which can be administered either separately i.e. by co-administration or in a fixed dose combination (FDC) in the treatment of gastrointestinal and related disorders.

### BACKGROUND OF THE INVENTION

Physicians have long recognized that conditions affecting the upper gastrointestinal (GI) tract commonly produce upper abdominal pain, discomfort, abdominal fullness, bloating, early satiety, nausea, vomiting, belching, heartburn and regurgitation. Such symptoms are typically postprandial and occur either alone or in combination. Overall, upper GI symptoms, including both dyspeptic-type and reflux-type, affect more than 25% of adults in the Western world and have a significant, negative impact on both functional status and sense of individual well-being (Tougas et al., Am J Gastroenterol. 1999).

Symptoms related to disorders of upper gut function are among the most common presenting complaints in primary-care and GI specialty medical practice. These disorders commonly include GERD (gastroesophageal reflux disease), GERD with erosion, NERD (non-erosive reflux disease), NUD (non-ulcer dyspepsia), PUD (peptic ulcer disease), FD (functional dyspepsia), diabetic gastroparesis, gastrointestinal ulcers, Zollinger-Ellison syndrome, and antral G-cell hyperplasia.

Gastro-esophageal reflux disease (GERD) is one of the most common diseases in Europe and the United States and affects severely the quality of life pertinent to such symptoms as heartburn and acid regurgitation. The prevalence of GERD referring to those with symptoms at least once per week, varies greatly with ethnicity and geography: 18.1-27.8% in North America, 8.8-25.9% in Europe, and 2.5-7.8% in East Asia, as estimated from 28 studies.

Gastroesophageal reflux is primarily a disorder of the lower esophageal sphincter (LES) but there are several factors that may contribute to its development. The factors influencing GERD are both physiologic and pathologic. The most common cause is transient lower esophageal sphincter relaxations (TLESRs). TLESRs are brief moments of lower esophageal sphincter tone inhibition that are independent of a swallow. While these are physiologic in nature, there is an increase in frequency in the postprandial phase and they contribute greatly to acid reflux in patients with GERD. Other factors include reduced lower esophageal sphincter (LES) pressure, hiatal hernias, impaired esophageal clearance and delayed gastric emptying.

GERD Treatment options include antacids-over-the-counter antacids are best for intermittent and relatively infrequent symptoms of reflux. When taken frequently, antacids may worsen the problem. They leave the stomach quickly, and your stomach increases acid production as a result.

Histamine 2 (H2) blockers are drugs that help lower acid secretion. H2 blockers heal esophageal erosions in about 50 percent of patients.

Proton pump inhibitors (PPIs) are drugs that block the three major pathways for acid production. PPIs suppress acid production much more effectively than H2 blockers.

Prokinetic/motility agents are drugs that enhance the activity of the smooth muscle of your gastrointestinal tract. These drugs are somewhat less effective than PPIs.

Current treatment options for management of GERD have their own limitations e.g. internationally, metoclopramide is an FDA approved Prokinetic/motility agent, however is known to cause extrapyramidal side effects. Levosulpiride is another drug used as a motility agent known to cause Gynecomastia. This adverse effect profile leads to non-compliance and poor quality of life. Domperidone is commonly used in combination with PPIs is very mild in terms of efficacy and one of the most effective motility agents. Cisapride has been withdrawn because of cardiac side effects.

Although dopamine is a major neurotransmitter in the central nervous system, it is also found in large concentrations in the gastrointestinal tract. Numerous physiological and pharmacological studies have shown that dopamine has an inhibitory effect on gastric motility. This inhibition is thought to be mediated by a decrease in acetylcholine release resulting from the stimulation of peripheral dopamine D2 receptors located on postganglionic cholinergic nerves. Indeed, it has been reported that the inhibition of gastric motility by dopamine is antagonized by the D2 receptor antagonist domperidone, but not by dopamine D1 receptor antagonists. Another hypothesis regarding the mechanism responsible for the effect of dopamine on gastric motility is that the inhibition by dopamine is mediated, at least in part, via the central dopaminergic system. Previous studies have shown that the delay in gastric emptying that is induced by dopamine receptor agonists is mainly inhibited by centrally acting dopamine D2 receptor antagonists and that central administration of apomorphine delays gastric emptying in guinea pigs.

Dopamine D2 receptors have been divided into three subtypes, based on their molecular structure and pharmacological properties: D2, D3 and D4 receptors. Of these receptor subtypes dopamine D3 receptors may, because of their high density in the mesolimbic dopaminergic projection field, play an important role in the pathogenesis of psychiatric disorders. In addition, dopamine D3 receptor agonists have been shown to produce sniffing and yawning behaviors, hypothermia, hypertensive effects and lead to reductions in basal gastric acid and pepsin secretion in rats and vomiting in dogs and ferrets. While the involvement of the dopamine D3 receptor in gastric motility is not well understood, recent studies have confirmed the presence of dopamine D3 receptors in the myenteric neurons of mouse and rat stomachs.

Drugs like metoclopramide, where antiemetic effect is the result of dopamine D2 receptor antagonism in the chemoreceptor trigger zone in the central nervous system. Because of central action on D2 receptors, extrapyramidal reactions are the most common acute side effect of metoclopramide with a reported incidence of 0.2%, but in the aged and young patients this incidence increases up to as high as 25%. In another study, it was demonstrated that AS-8112 which is a potent dopamine D2, D3 and 5-HT3 receptors antagonist and is a highly potent antiemetic against emesis induced by various emetogens.

Metoclopramide also showed a broad-spectrum anti-emetic activity in this study. However, anti-emetic effects of metoclopramide were less potent than that of AS-8112. Furthermore, cisplatin- and morphine-induced emetic response was not completely reduced by metoclopramide at high doses (1±3 mg kg71 s.c. or i.v.). Metoclopramide at high doses (3±10 mg kg71 i.v.) also caused central nervous depression which was related to the blockade of dopamine D2 receptors. On the other hand, AS-8112 at doses that perfectly reduced emetic responses did not cause central nervous depression, catalepsy and extrapyramidal syndrome in rats and monkeys. Mesdopetam is under evaluation with USFDA for the treatment of levodopa-induced dyskinesia in Parkinson's disease (PD-LIDs).

In particular, the slow onset of action is an intrinsic limitation of all exiting PPIs used as monotherapy in GERD as it is strictly linked to the pharmacokinetics and mode of action of all PPIs. After absorption and distribution PPIs given their pKa, accumulate in the acid space of the canaliculus of secreting parietal cell, where they are transformed into the active Sulphenamide which forms non-competitive, covalent, and irreversible bonds with the key cysteines of the H′, K-APTase (Sachs G et al. Annu Rev Pharmacol Toxicol 1995; 35:277-305).

Due to the irreversible nature of this binding, a steady-state inhibition is achieved only after 3 or 4 days of treatment. This can be attributed to their very short half-life in combination with an activation of over 75% of the pumps and constant pump turnover in the face of covalent inhibition of the pump (Sachs G. Eur J. Gastroenterol Hepatol. 2001; 13 (Suppl. 1): S35-S41).

U.S. Pat. No. 7,846,921 discloses method for relieving or ameliorating symptoms of gastroesophageal reflux disease by administering a medicament that includes the cholecystokin-2 (CCK-2) receptor antagonist itriglumide and a proton pump inhibitor (PPI) for the treatment of patients suffering from gastrointestinal or related disorders.

IN248777 discloses a pharmaceutical composition, wherein a combination of enteric coated proton pump inhibitor with modified release domperidone is formulated. The said combination is effective in the treatment of gastrointestinal disorders, mainly gastroesophageal reflux disease and dyspepsia.

WO 2012143337 discloses phenoxy-ethyl-amine derivatives useful as modulators of cortical and basal ganglia dopaminergic and N-methyl-D-aspartate (NMDA) receptor mediated glutamatergic neurotransmission, and more specifically for the treatment of diseases that are responsive to modulation of dopaminergic and glutamatergic function in the central nervous system. The compound [2-(3-fluoro-5-methanesulfonylphenoxy)-ethyl](propyl)amine is disclosed in its non-salt form as well as in the form of a hydrochloric acid salt in Example 1. It is stated that hydrochloric acid salt has a melting point of 191° C.

WO2020239568A1 discloses tartaric acid and fumaric acid salts of Mesdopetam.

Considering the drawbacks associated with current therapy for the treatment of gastrointestinal disorders, there is a need to develop novel treatment options for patients with gastrointestinal disorders, which can overcome the drawbacks in the currently available treatment therapies.

### SUMMARY OF THE INVENTION

Surprisingly, inventors of present invention have found that Mesdopetam alone or in combination with a PPIs offers a unique advantage among all anti-secretive drugs due to their mode of action, as Mesdopetam stimulates gastric motility via inhibition of Dopamine D2/D3 receptors and Alpha-1 adrenergic receptors and proton pump inhibitors (PPIs) inhibits the secretion of the gastric acid by irreversibly blocking the enzyme system H⁺K⁺ ATPase of the gastric parietal cell, thus prevents secretion of gastric acid.

None of the prior art reveals composition comprising Mesdopetam alone or in combination with proton-pump inhibitors for the treatment of gastrointestinal and related disorders.

After screening 300 molecules using various genome based and receptor based activity to target motility in GERD, about 80 were filtered. Subsequently, based on deeper understanding of molecular mechanism and disease pathway correlation, Mesdopetam was amongst those where mechanistic reconstruction was applied. After extensive study of receptor mechanism, pathways, disease patho-physiology and molecular mechanism crisscross with disease biochemical/biomarker/receptor signaling/down-signaling activity, Mesdopetam is found to be one of the most promising candidates for the management of GERD.

Mesdopetam stimulates gastric motility via inhibition of Dopamine D2/D3 receptors and Alpha-1 adrenergic receptors. It is expected to have predominant action with plausible improvement in gastric motility and would fill the gap for Cisapride and other motility agents. Proton pump inhibitors (PPIs) inhibit the secretion of the gastric acid by irreversibly blocking the enzyme system H⁺K⁺ ATPase of the gastric parietal cell and thus prevents secretion of gastric acid. Examples of PPIs which can be used along with Mesdopetam includes, but are not limited to Pantoprazole, Omeprazole, Esomeprazole, Rabeprazole, Lansoprazole, Ilaprazole Dexlansoprazole and the like.

Accordingly, the present invention relates to a composition comprising Mesdopetam for the treatment of gastrointestinal disorders.

In an aspect the present invention provides a pharmaceutical composition for managing gastrointestinal disorders comprising:
a. mesdopetam or a pharmaceutically acceptable salt thereof; and
b. at least one pharmaceutically acceptable carrier or excipients.

In another aspect the present invention provides a pharmaceutical composition, wherein mesdopetam is present in amount in a range of 1 to 40% w/w of the total weight of the pharmaceutical composition.

In another aspect the present invention provides a pharmaceutical composition for managing gastrointestinal disorders comprising:
a. mesdopetam or a pharmaceutically acceptable salt;
b. proton pump inhibitor; and
c. at least one pharmaceutically acceptable carrier or excipients.

In another aspect the present invention provides a pharmaceutical composition, wherein mesdopetam is present in amount in a range of 1 to 40% w/w and proton pump inhibitor is present in amount in a range of 1 to 50% w/w of the total weight of the pharmaceutical composition.

In another aspect the present invention provides a pharmaceutical composition, wherein the pharmaceutical composition is in the form of a capsule, a tablet, suspension and pellets.

In another aspect the present invention provides a pharmaceutical composition, wherein the pharmaceutically acceptable excipients or carriers is selected from one or more diluents, binders, controlled release polymer, lubricants, glidants, disintegrants, coating materials and solvents or combination thereof.

In another aspect the present invention provides a pharmaceutical composition, wherein the controlled release polymer is selected from Hydroxypropyl methyl cellulose, Hydroxypropyl cellulose, Ethyl cellulose, Pectin, macrogol, Hydrogenated castor oil, Carbopol, Methyl methacrylate, Polyvinyl pyrrolidine, shellac or combination thereof.

In another aspect the present invention provides a pharmaceutical composition, wherein Mesdopetam is in the form of immediate or modified release (MR) dosage forms.

In another aspect the present invention provides a pharmaceutical composition, wherein the at least one proton pump inhibitor is chosen from Pantoprazole, Omeprazole, Esomeprazole, Rabeprazole, Lansoprazole, Ilaprazole, Dexlansoprazole or combination thereof.

In another aspect the present invention provides a pharmaceutical composition, wherein the composition is in the form of modified release (MR) dosage forms, wherein modified release dosage form is enteric coated dosage form selected form delayed release capsule, delayed release pellets/granules filled into capsule, delayed release granules for suspension, delayed release tablet, and delayed release orally disintegrating tablet.

In another aspect the present invention provides a pharmaceutical composition as disclosed herein or mesdopetam, for use in the treatment of gastrointestinal disorders in a subject in need thereof.

In another aspect the present invention provides a pharmaceutical composition as disclosed herein or mesdopetam, for use in the treatment of gastrointestinal disorders in a subject in need thereof, wherein the gastrointestinal disorder is GERD (Gastroesophageal Reflux Disease), GERD with erosion, NERD (Non-Erosive Reflux Disease), NUD (Non-Ulcer Dyspepsia), PUD (Peptic Ulcer Disease), FD (Functional Dyspepsia), Diabetic Gastroparesis, Nocturnal heartburn, Heartburn, Bloating, gastrointestinal ulcers, Zollinger-Ellison syndrome, erosive esophagitis and antral G-cell hyperplasia or a combination thereof.

In another aspect the present invention provides use of mesdopetam or pharmaceutical composition as disclosed herein, in the manufacture of a medicament for the treatment of gastrointestinal disorders in a subject in need thereof wherein the medicament is to be administered to the subject.

In another aspect the present invention provides a use of mesdopetam or pharmaceutical composition in treating gastrointestinal disorders, wherein the gastrointestinal disorder is GERD (Gastroesophageal Reflux Disease), GERD with erosion, NERD (Non-Erosive Reflux Disease), NUD (Non-Ulcer Dyspepsia), PUD (Peptic Ulcer Disease), FD (Functional Dyspepsia), Diabetic Gastroparesis, Nocturnal heartburn, Heartburn, Bloating, gastrointestinal ulcers, Zollinger-Ellison syndrome, erosive esophagitis and antral G-cell hyperplasia or a combination thereof.

In another aspect the present invention provides a method of treating gastrointestinal diseases in a subject in need thereof comprising administering to the subject mesdopetam and pharmaceutical composition as disclosed herein, comprising a therapeutically effective amount of mesdopetam or a pharmaceutically acceptable salt thereof.

In another aspect the present invention provides a method of treating gastrointestinal disorders, wherein the gastrointestinal disorders is GERD (Gastroesophageal Reflux Disease), GERD with erosion, NERD (Non-Erosive Reflux Disease), NUD (Non-Ulcer Dyspepsia), PUD (Peptic Ulcer Disease), FD (Functional Dyspepsia), Diabetic Gastroparesis, Nocturnal heartburn, Heartburn, Bloating, gastrointestinal ulcers, Zollinger-Ellison syndrome, erosive esophagitis and antral G-cell hyperplasia or a combination thereof.

In another aspect the present invention provides the method as disclosed herein, wherein mesdopetam alone or in combination with proton pump inhibitor can be administered as once daily (QD).

In another aspect the present invention provides the method as disclosed herein, wherein mesdopetam alone or in combination with proton pump inhibitor can be administered as twice a day (BID), thrice a day (TID) or four times a day (QID).

In another aspect the present invention provides the method as disclosed herein, wherein pharmaceutical composition comprising mesdopetam is administered in a dose range of 0.05 mg to 8000 mg per day, preferably 0.5 mg to 7500 mg per day and more preferably 1 mg to 7200 mg per day.

In another aspect the present invention provides the method as disclosed herein, wherein pharmaceutical composition comprising mesdopetam is administered in a dose range of 10 mg to 160 mg per day for the first two weeks and then gradually increased to 20 mg to 320 mg per day of mesdopetam per day and the dose of mesdopetam can be adjusted according to the patient's age and body weight.

In another aspect the present invention provides the method as disclosed herein, wherein pharmaceutical composition comprising mesdopetam is administered in dose range of 0.05 mg to 8000 mg per day, preferably 0.5 mg to 7500 mg per day and proton pump inhibitor with dose range of 1 mg to 300 mg per day, preferably 2.5 mg to 240 mg per day, more preferably 10 mg to 100 mg per day.

In another aspect the present invention provides the method as disclosed herein, wherein pharmaceutical composition comprising mesdopetam is administered in a dose range of 10 mg to 160 mg per day for the first two week and then gradually increased to 20 to 320 mg of mesdopetam per day and proton pump inhibitor in a dose range of 1 mg to 300 mg per day, preferably 2.5 mg to 240 mg per day, more preferably 10 mg to 100 mg per day.

In another aspect the present invention provides the method as disclosed herein, wherein pharmaceutical composition comprising mesdopetam alone or in combination with proton pump inhibitor, can be administered to patient population such as pediatric, adult or geriatric.

### DETAILED DESCRIPTION OF THE INVENTION

The present invention can be more readily understood by reading the following description of the invention and study of the included examples.

As used herein the term “active ingredient” or “active agent” or “drug” as used herein means Mesdopetam or PPIs and its salt, solvate, or some additional actives as described herein or combination thereof that induce a desired pharmacological or physiological effect.

The term “derivatives” includes all pharmaceutically acceptable salts, solvates, esters, isomers, polymorphs of Mesdopetam or PPIs and can be used interchangeably.

The term “pharmaceutical composition” or “composition” and the like are used interchangeably, and used herein refers to a solid dosage form comprising Mesdopetam alone or in combination with proton pump inhibitors (PPIs) suitable for oral administration such as tablets e.g. enteric coated tablets, bilayer tablets, film coated tablets, etc., capsule, granules, powders, premix, micro-tablets, MUPS (Multiple Unit Pellet System), orally disintegrating tablets (ODT), solution, suspension, emulsion, elixir, syrup, and the like.

The term “w/w” refers to total weight of substance/excipients with respect to total composition weight or the proportion of a particular substance within a mixture, as measured by weight or mass.

The term “about” as used herein means a deviation within 10%, preferably within 5%, and even more preferably, within 2% of the numbers reported.

The term “stable” refers to formulations that substantially retain the labelled amount of the therapeutically active ingredient during storage for commercially relevant times, and the drug-related impurity contents in the formulations remain within acceptable limits.

As used herein, unless otherwise specified, the term “patient” or “subject” refers to a human patient unless indicated otherwise.

As used herein, unless otherwise specified, the terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient respond to the disease or disorder.

The term “therapeutically effective amount” is defined to mean the amount or quantity of the active drug is sufficient to elicit an appreciable pharmacological response, when administered to the patient.

The use of the word “a” or “an” when used in conjunction with the term “comprising” in the claims and/or the specification may mean “one,” but it is also consistent with the meaning of “one or more,” “at least one,” and “one or more than one.”

In one aspect, the present invention relates to a composition comprising Mesdopetam for the treatment of gastrointestinal disorders.

In one of the embodiments, the present invention relates to a pharmaceutical composition comprising Mesdopetam for the treatment of gastrointestinal disorders.

In an another embodiment, the present invention relates to a pharmaceutical composition comprising Mesdopetam alone or in combination with proton pump inhibitor for the treatment of gastrointestinal disorders.

In an another embodiment, the present invention relates to a pharmaceutical composition comprising Mesdopetam alone or in combination with proton pump inhibitors (PPIs), which can be administered either separately i.e. by co-administration or in a fixed dose combination (FDC) in the treatment of gastrointestinal disorders.

In an another embodiment, the present invention relates to a pharmaceutical composition used for treatment of gastrointestinal disorders comprising Mesdopetam alone or in combination with a proton pump inhibitor (PPIs) along with a pharmaceutically acceptable carrier or excipient, wherein Mesdopetam and PPIs are present at therapeutically effective dosages to provide desired therapeutic effect.

In an another embodiment, the pharmaceutical composition comprising Mesdopetam is in the form of immediate and/or modified release (MR) dosage forms and PPIs is in the form of modified release (MR) dosage forms, particularly enteric coated dosage form such as delayed release capsule, delayed release pellets/granules filled into capsule, delayed release granules for suspension, delayed release tablet, and delayed release orally disintegrating tablet.

In an another embodiment, the pharmaceutical composition comprising Mesdopetam is in the form of immediate or modified release (MR) dosage forms.

In an another embodiment, the pharmaceutical composition comprising PPIs in the form of modified release (MR) dosage forms, particularly enteric coated dosage form such as delayed release capsule, delayed release pellets/granules filled into capsule, delayed release granules for suspension, delayed release tablet, and delayed release ODT.

In an another embodiment, the pharmaceutical composition comprising Mesdopetam alone in the form of immediate and/or modified release composition or in combination with PPIs as conventional or MR tablets, can be filled into a capsule, can be made as bilayer tablets, other dosage forms suitable for combination therapy and can be developed as kit of individual active ingredients.

In an another embodiment, the pharmaceutical composition comprising combination of Mesdopetam alone or in combination with proton pump inhibitors (PPIs) is suitable for delivery as a topical dosage form such as emulsion, ointment, cream, lotion, gel, paste, transdermal delivery gel and patch, etc.

In an another embodiment, the pharmaceutical composition comprising combination of Mesdopetam alone or in combination with proton pump inhibitors (PPIs) is suitable for delivery as a parenteral dosage form such as a solution, suspension, emulsion, and dry powder for reconstitution, infusion, etc. which can be administered by intravenous, intramuscular and subcutaneous route.

In an another embodiment, the pharmaceutical composition comprising combination of Mesdopetam and proton pump inhibitors (PPIs) is either separated from each other or formulated into a single composition.

In another aspect, the said pharmaceutical composition can be in the form of immediate release and/or modified release (MR) or combination thereof.

The term modified-release drug product as used herein is to describe products that alter the timing and/or the rate of release of the drug substance.

The pattern of drug release from modified-release (MR) dosage forms is deliberately changed from that of a conventional (immediate-release) dosage formulation to achieve a desired therapeutic objective or better patient compliance. Types of MR drug products include delayed release (e. g, enteric coated), extended release (ER), and orally disintegrating tablets (ODT). Extended-release drug product is a dosage form that allows at least a twofold reduction in dosage frequency as compared to that drug presented as an immediate-release (conventional) dosage form. Examples of extended-release dosage forms include controlled-release, sustained-release, long-acting drug products and the like.

Delayed-release drug product is a dosage form that releases a discrete portion or portions of drug at a time other than promptly after administration. An initial portion may be released promptly after administration. Enteric-coated dosage forms are common delayed-release products.

Targeted-release drug product is a dosage form that releases drug at or near the intended physiologic site of action. Targeted-release dosage forms may have either immediate- or extended-release characteristics.

“Pharmaceutically acceptable excipient(s)” are components that are added to the pharmaceutical composition other than the active ingredient. Pharmaceutically acceptable excipient(s) includes, but are not limited to, diluent, release modifier or controlled release polymer, Enteric coating agent, binder, disintegrant, lubricant/glidants, antioxidant, plasticizer, surfactants, solvent/vehicle, flavors or colorants, coating materials and any other excipient known to the art for making pharmaceutical composition for oral administration such as tablets e. g. enteric coated tablets, bilayer tablets, film coated tablets, etc., capsule, granules, powders, premix, micro-tablets, MUPS (Multiple Unit Pellet System), orally disintegrating tablets (ODT), solution, suspension, emulsion, elixir, syrup, and the like.

Diluents according to the present invention are selected from, but are not limited to, Light magnesium oxide, silicon dioxide, titanium dioxide, talc, celluloses, microcrystalline cellulose, starch, mannitol, sorbitol or other sugar alcohols, sucrose, lactose, and the like used either alone or in combinations thereof.

Release modifiers or controlled release polymer according to the present invention are selected from, but are not limited to, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanth, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose, cellulose acetate, cellulose acetate butyrate and ethyl cellulose; and other materials known to those of ordinary skill in the art. Other suitable release modifiers that can be used for this purpose include polyvinylalcohol, polyethylene oxide, a blend of gelatin and polyvinylpyrrolidone, gelatin, glucose, saccharides, copovidone, poly(vinyl) pyrrolidone)-polyvinyl acetate) copolymer, Acryl-EZE®, Eudragit® NE, RL and RS, hydroxypropylcellulose, microcrystalline cellulose, polyethylene-vinyl acetate copolymer, 2-hydroxyethylmethacrylate (HEMA), methyl methacrylate (MMA), calcium pectinate, macrogol, hydrogenated castor oil, Carbopol, shellac and the like. Composition according to the present invention are either mixed or coated with above release modifiers.

Enteric coating agents according to the present invention are selected from, but are not limited to, polymethylmethacrylic acid esters (Eudragit), cellulose acetate phthalate (CAP), Cellulose acetate succinate, Hydroxypropyl methyl cellulose phthalate, Hydroxypropyl methyl cellulose acetate succinate, Polyvinyl acetate phthalate (PVAP), Cellulose acetate trimellitate, Shellac, Zein, and Sodium alginate.

Binders according to the present invention are selected from, but are not limited to, natural such as starch, pregelatinized starch, Sodium alginate, and gelatin, etc. Synthetic/Semisynthetic such as Polyvinyl Pyrrolidone (PVP), Methylcellulose, Hydroxy Propyl Methyl Cellulose (Hypromellose), Polymethacrylates, Sodium Carboxy Methyl Cellulose, Polyethylene Glycol (PEG), Macrogol 400 and Methylcellulose, etc. Saccharides and their derivatives such as Disaccharides such as lactose and sucrose, Polysaccharides and their derivatives such as starches, cellulose or modified cellulose such as MCC and cellulose ethers such as hydroxypropyl cellulose (HPC), Sugar derivatives such as sorbitol, xylitol, and mannitol, and the like used either alone or in combinations thereof.

Disintegrants according to the present invention are selected from, but are not limited to, L-HPC (Low-Substituted Hydroxy propyl Cellulose), croscarmellose sodium, crospovidone, sodium starch glycolate, polacrilin potassium, microcrystalline cellulose, starches like used either alone or in combinations thereof.

Lubricants according to the present invention are selected from, but are not limited to, magnesium stearate, calcium stearate, stearic acid, sodium stearyl fumarate, and the like used either alone or in combinations thereof.

Glidants according to the present invention are selected from, but are not limited to, colloidal silicon dioxide (Aerosil), calcium silicate, magnesium silicate, silicon hydrogel, cornstarch, talc, and the like used either alone or in combinations thereof.

Antioxidants are used to prevent oxidation, thereby preventing the deterioration of the preparation. Suitable antioxidants for use in the present invention include but are not limited to propyl gallate, butylated hydroxyanisole (BHA), butylated hydroxytoluene (BHT), and combinations thereof. Suitable preservatives that may be added to the composition include but are not limited to sodium benzoate, potassium benzoate, Benzyl alcohol, Methyl Paraben, Ethyl Paraben, Propyl Paraben, and the like used either alone or in combinations thereof. Plasticizer refers to an excipient that can impart flexibility and/or stretchability to a coat or membrane. Examples of plasticizers include but are not limited to Dibutyl phthalate, medium chain triglycerides, phthalates, phosphates, glycerol, citrates, adipates, sebacates, succinates, glycolates, Triethyl citrate and the like used either alone or in combinations thereof.

Surfactant refers to one or more excipients that may be added to the pharmaceutical composition to facilitate dissolution of poorly soluble excipients and/or to increase dissolution rate of composition or components thereof. Examples of surfactants include but are not limited to sodium lauryl sulphate, glycerol monostearate, sorbitan monolaurate, tween 60, tween 80 (polysorbate 80) etc.

A solvent/vehicle is a substance that dissolves a solute (a chemically distinct liquid, solid or gas), resulting in a solution. Examples include but are not limited to purified water, alcoholic solvents methanol, ethanol; Isopropyl Alcohol, ketones such as acetone, propanone; esters such as ethyl acetate, n-propyl acetate, isopropylacetate and n-butyl acetate and the like; ethers such as dimethylether, diethylether, methyltertiarybutylether, ethylmethylether, diisopropylether, dichloromethane and dioxane.

Flavorants refers to a substance that gives another substance flavor, altering the characteristics of the solute, causing it to become sweet, sour, etc. Pharmaceutically acceptable flavors include but are not limited to cherry, orange, vanilla and the like.

Tablet coating refers to the phenomenon of application of coating to the tablet, which involves some coating agents like plasticizer, polymers, colorants and solvent.

Pharmaceutically acceptable acid includes but are not limited to citric acid, tartaric acid, succinic acid, malic acid, fumaric acid, salicylic acid, benzoic acid, cinnamic acid or adipic acid. Colorants is a substance that is preferably added to the coating agent for coating of the tablet. Pharmaceutically acceptable colorants include but are not limited to red iron oxide, non-irradiated, iron oxide black, Ferrosoferric oxide, tartrazine, erythrosine, amaranth lake, opadry systems, titanium dioxide, and iron oxide yellow.

Mesdopetam is a novel dopamine D3 receptor antagonist, and has the chemical name-[2-(3-fluoro-5-methylsulfonylphenoxy) ethyl]propan-1-amine. The chemical structure of Mesdopetam is as follows:

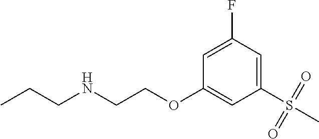 [SMILES: CCCNCCOc1cc(F)cc(S(C)(=O)=O)c1]

Mesdopetam acts by antagonizing the dopamine D3 receptor and thereby counteracting the physiological effects of the signal substance dopamine. The dopamine D3 receptor is genetically linked to increased risk of involuntary movements and patients with PD-LIDs have higher amounts of D3 receptors in parts of the brain essential for the control of movements.

Mesdopetam is a novel compound with psychomotor stabilizing properties primarily targeting the dopamine D3 receptor. It is a novel dopamine D3 receptor antagonist. Mesdopetam is developed as an experimental treatment for levodopa-induced dyskinesia (LID), impulse control disorder, and psychosis in Parkinson's disease (PD). IRL790 exerts its efficacy primarily via interaction with D3R (antagonist, Ki about 90 nM) and as such may be useful to treat not only LID but also several other complications of therapy in PD, where D3R dysfunction is implicated.

In experimental animal models, Mesdopetam displays both antidyskinetic and antipsychotic properties, while leaving normal behavior largely unaffected, indicating a novel pharmacological profile with the potential to alleviate several troubling complications of therapy commonly seen in the management of parkinsonian. Mesdopetam is metabolized to two main pharmacologically inactive metabolites in man; the dealkylated M1 (IRL902) via CYP450, which is further acetylated by N-acetyltransferase 2 (NAT2) to M2 (IRL872). Both metabolites are present in plasma and urine.

In an another embodiment, the present invention relates to a composition used for treatment of gastrointestinal disorders, where the gastrointestinal disorders includes GERD (Gastroesophageal Reflux Disease), GERD with erosion, NERD (Non-Erosive Reflux Disease), NUD (Non-Ulcer Dyspepsia), PUD (Peptic Ulcer Disease), FD (Functional Dyspepsia), Diabetic Gastroparesis Nocturnal heartburn, Heartburn, Bloating, gastrointestinal ulcers, Zollinger-Ellison syndrome, erosive esophagitis and antral G-cell hyperplasia.

In an another embodiment, the present invention relates to a composition used for the treatment of gastrointestinal disorders where the gastrointestinal disorders include short term treatment of erosive esophagitis associated with GERD and maintenance of healing of erosive esophagitis.

In an another embodiment, the present invention relates to a composition of Mesdopetam which can be given as monotherapy or as combination therapy along with PPIs for the treatment of Gastroesophageal Reflux Disease (GERD).

In an another embodiment, the present invention relates to a pharmaceutical composition comprising Mesdopetam alone or in combination with proton pump inhibitor for the treatment of Gastroesophageal Reflux Disease (GERD).

In an another embodiment, the invention relates to use of Mesdopetam alone or in combination with proton pump inhibitor for the treatment of Gastroesophageal Reflux Disease (GERD).

In an another embodiment, the present invention relates to a composition used for treating Gastroesophageal Reflux Disease (GERD) using Mesdopetam alone or in combination with proton pump inhibitor.

In an another embodiment, the present invention relates to a pharmaceutical composition comprising Mesdopetam alone or in combination with proton pump inhibitor which can be administered as once daily (QD).

In an another embodiment, the present invention relates to a pharmaceutical composition comprising Mesdopetam alone or in combination with proton pump inhibitor, which can be administered twice a day (BID), thrice a day (TID) or four times a day (QID).

In an another embodiment, present invention relates to pharmaceutical composition comprising Mesdopetam alone or in combination with proton pump inhibitor which can be administered to patient population such as pediatric, adult or geriatric.

In an another embodiment, the present invention relates to a pharmaceutical composition comprising combination of Mesdopetam and a proton pump inhibitor, where the proton pump inhibitor is selected from group consisting of Omeprazole, Pantoprazole, Esomeprazole, Rabeprazole, Lansoprazole, Ilaprazole and Dexlansoprazole or combination thereof.

In an another embodiment, the present invention relates to a pharmaceutical composition comprising Mesdopetam administered in a dose range of 0.05 mg to 8000 mg per day, preferably 0.5 mg to 7500 mg per day and more preferably 1 mg to 7200 mg per day.

The present invention provides a pharmaceutical composition comprising mesdopetam in an amount of about 2 to about 100 mg, preferably about 3 to about 50 mg, more preferably about 6 mg, 12 mg, 36 mg, 48 mg.

In an another embodiment, the present invention relates to a pharmaceutical composition comprising mesdopetam or pharmaceutically acceptable salt thereof in an amount of about 1 to about 40% w/w, preferably about 2 to about 20% w/w, more preferably about 2 to 10% w/w of the total composition.

In an another embodiment, the present invention relates to a pharmaceutical composition comprising Mesdopetam administered in a dose range of 10 mg to 160 mg per day for the first two weeks and then gradually increased to 20 mg to 320 mg per day of Mesdopetam per day and the dose of Mesdopetam can be adjusted according to the patient's age and body weight.

In an another embodiment, the present invention relates to a pharmaceutical composition comprising proton pump inhibitor (PPIs) administered in a dose range of 1 mg to 300 mg per day, preferably 2.5 mg to 240 mg per day, more preferably 10 mg to 100 mg per day and the dose of PPIs can be adjusted according to the patient's age and body weight.

In an another embodiment, the present invention relates to a pharmaceutical composition comprising PPIs in an amount of about 1 to about 50% w/w, preferably about 2 to about 25% w/w, more preferably about 2 to 10% w/w of the total composition.

In an another embodiment, the present invention relates to a pharmaceutical composition comprising Mesdopetam with dose range of 0.05 mg to 8000 mg per day, preferably 0.5 mg to 7500 mg per day and proton pump inhibitor with a dose range of 1 mg to 300 mg per day, preferably 2.5 mg to 240 mg per day, more preferably 10 mg to 100 mg per day.

In an another embodiment, the present invention relates to a pharmaceutical composition comprising Mesdopetam with dose range of 10 mg to 160 mg per day for the first two weeks and then gradually increased to 20 mg to 320 mg per day and proton pump inhibitor with dose range of 1 mg to 300 mg per day, preferably 2.5 mg to 240 mg per day, more preferably 10 mg to 100 mg per day.

In an another embodiment, the present invention relates to a pharmaceutical composition comprising combination of Mesdopetam with dose range of 0.05 mg to 8000 mg per day and proton pump inhibitors with dose range of Pantoprazole 20 mg to 240 mg per day, Omeprazole 10 mg to 40 mg per day, Esomeprazole 2.5 mg to 240 mg per day, Rabeprazole 5 mg to 40 mg per day, Lansoprazole 10 mg to 60 mg per day, Ilaprazole 5 mg to 20 mg per day, and Dexlansoprazole 20 mg to 80 mg per day.

In an another embodiment, the present invention relates to a pharmaceutical composition comprising combination of Mesdopetam in a dose range of 0.05 mg to 8000 mg per day and Pantoprazole with a dose range of 20 mg to 240 mg per day, preferably 40 mg once daily.

In an another embodiment, the present invention relates to a pharmaceutical composition comprising combination of Mesdopetam in a dose range of 0.05 mg to 8000 mg per day and buffered pantoprazole with a dose range of 20 mg to 240 mg per day, preferably 40 mg once daily.

In an another embodiment, the present invention relates to a pharmaceutical composition comprising combination of Mesdopetam in a dose range of 0.05 mg to 8000 mg per day and Omeprazole with a dose range of 10 mg to 40 mg per day, preferably 10 mg, 20 mg once daily.

In an another embodiment, the present invention relates to a pharmaceutical composition comprising combination of Mesdopetam in a dose range of 0.05 mg to 8000 mg per day and buffered omeprazole with a dose range of 10 mg to 40 mg per day, preferably 10 mg, 20 mg once daily.

In an another embodiment, the present invention relates to a pharmaceutical composition comprising combination of Mesdopetam in a dose range of 0.05 mg to 8000 mg per day and Esomeprazole with a dose range of 2.5 mg to 240 mg per day, preferably 2.5 mg, 5 mg, 10 mg, 20 mg, 40 mg once daily.

In an another embodiment, the present invention relates to a pharmaceutical composition comprising combination of Mesdopetam in a dose range of 0.05 mg to 8000 mg per day and Rabeprazole with a dose range of 5 mg to 40 mg per day, preferably 5 mg, 10 mg, 20 mg once daily.

In an another embodiment, the present invention relates to a pharmaceutical composition comprising combination of Mesdopetam in a dose range of 0.05 mg to 8000 mg per day and Lansoprazole with a dose range of 10 mg to 60 mg per day, preferably 15 mg, 30 mg, 60 mg once daily.

In an another embodiment, the present invention relates to a pharmaceutical composition comprising combination of Mesdopetam in a dose range of 0.05 mg to 8000 mg per day and Ilaprazole with a dose range of 10 mg to 40 mg per day, preferably 5 mg, 10 mg, 20 mg once daily.

In an another embodiment, the present invention relates to a pharmaceutical composition comprising combination of Mesdopetam in a dose range of 0.05 mg to 8000 mg per day and Dexlansoprazole with a dose range of 20 mg to 80 mg per day, preferably either 30 mg or 60 mg once daily.

The composition of the Mesdopetam alone or in combination with proton pump inhibitors (PPIs) can be packed into suitable packaging system e. g. Blister packs, strip packs, alu-alu packs, bottles such as glass and plastic bottles, sachet packaging, etc.

### EXAMPLES

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### Example 1: Mesdopetam Modified Release Composition

TABLE 1
Composition.

| Sr. No. | Ingredients | Ranges in % (w/w) |
|---|---|---|
| 1. | Mesdopetam | 10-20 |
| 2. | Fumaric acid | 5-50 |
| 3. | Succinic acid | 5-50 |
| 4. | Citric acid | 5-50 |
| 5. | Hypromellose | 5-30 |
| | (Methocel K 100M) | |
| 6. | Hypromellose | 0-10 |
| | (Methocel K 15M) | |
| 7. | Povidone K30 | 1-5 |
| 8. | Isopropyl Alcohol | Q.S |
| 9. | Lactose anhydrous | 2-40 |
| 10. | Magnesium stearate | 0.5-1 |
| 11. | Hypromellose 5 cps | 0.5-2 |
| 12. | Macrogol 400 | 0.05-1 |
| 13. | Isopropyl alcohol* | Q, S |
| 14. | Dichloromethane* | Q, S |
| 15. | Methacrylic acid copolymer | 1-5 |
| 16. | Triethyl citrate | 0.1-1 |
| 17. | Talc | 0.1-1 |
| 18. | Titanium Dioxide | 0.1-1 |
| 19. | Colorants | Q, S |
| 20. | Isopropyl Alcohol* | Q, S |

*Evaporates during processing

Brief manufacturing process:
1. Mesdopetam, fumaric acid and hydroxypropyl methyl cellulose sifted through suitable mesh, followed by mixing and granulating with a solution of polyvinylpyrrolidone (K-30) in isopropyl alcohol.
2. Drying and grading of the granules through appropriate mesh.
3. Lubricating the granules with Lactose and Magnesium Stearate and form modified release composition either by compressing the granules, making the pellets.
4. Seal-coating the modified release composition of step 3 with hypromellose and optionally enteric coating with polymers such as Eudragit.

### Example 2: Mesdopetam Immediate Release Composition

TABLE 2
Composition

| Sr. No. | Ingredients | Ranges in % (w/w) |
|---|---|---|
| 1. | Mesdopetam | 10-20 |
| 2. | Lactose anhydrous | 10-60 |
| 3. | Microcrystalline cellulose | 10-60 |
| 4. | Low substituted Hydroxy | 5-20 |
| | propyl cellulose | |
| 5. | Sodium starch glycolate | 2-10 |
| 6. | Croscarmellose sodium | 2-10 |
| 7. | Colloidal anhydrous | 0.1-1 |
| | silica (Aerosil) | |
| 8. | Talc | 0.5-2 |
| 9. | Magnesium Stearate | 0.5-2 |

### Brief Manufacturing Process:

1 Mesdopetam, lactose, microcrystalline cellulose, hydroxypropyl cellulose, sodium starch glycolate and cross carmellose sodium sifted through appropriate mesh followed by mixing.
2. Magnesium stearate, colloidal silicon-di-oxide and talc sifted through appropriate mesh and mixed with blend of step 1.
3. Mesdopetam immediate release composition such as pellets, tablets, and granules obtained by using blend of step 2.

### Example 3: Mesdopetam Extended-Release Tablets 6 mg

TABLE 3
Composition

| Ingredients | mg/tablet | % w/w |
|---|---|---|
| Mesdopetam | 6 | 2.25 |
| Hydroxypropyl | 200 | 74.3 |
| methyl cellulose | | |
| Microcrystalline | 50 | 18.5 |
| cellulose | | |
| Talc/Colloidal anhydrous | 3 | 1.1 |
| silica (Aerosil) | | |
| Magnesium stearate | 5 | 1.8 |
| Blend of coating aids | 5 | 1.8 |
| Total weight/tablet (mg) | 269 | 100 |

### Brief Manufacturing Process:

1. Mesdopetam, Hydroxypropyl methyl cellulose, and Microcrystalline cellulose were mixed in a blender for 20 min.
2. Talc/Aerosil and Magnesium stearate were added in step 1 and mixed for 5 min.
3. Mixture obtained in step 2 was compressed using 5 mm punch.
4. The tablets were coated with the pharmaceutically acceptable coating aids.

### Example 4: Mesdopetam & Pantoprazole Extended-Release Tablets

TABLE 4
Composition

| Ingredients | mg/tablet | % w/w |
|---|---|---|
| Mesdopetam | 6 | 2.25 |
| Pantoprazole | 20 | 7.43 |
| Hydroxypropyl | 180 | 66.9 |
| methyl cellulose | | |
| Microcrystalline | 50 | 18.5 |
| cellulose | | |
| Talc/Colloidal anhydrous | 3 | 1.1 |
| silica (Aerosil) | | |
| Magnesium stearate | 5 | 1.8 |
| Blend of coating aids | 5 | 1.8 |
| Total weight/tablet (mg) | 269 | 100 |

### Brief Manufacturing Process:

1. Mesdopetam, Pantoprazole, hydroxypropyl methyl cellulose, and microcrystalline cellulose were mixed in a blender for 20 min.
2. Talc/Aerosil and magnesium stearate were added in step 1 and mixed for 5 min.
3. Mixture obtained in step 2 was compressed using 5 mm punch.
4. The tablets were coated with the pharmaceutically acceptable coating aids.

### Example 5: Enteric Coated PPIs

TABLE 5
Composition

| Sr. No. | Ingredients | Ranges in % (w/w) |
|---|---|---|
| 1. | Pantoprazole | 1-20 |
| 2. | Omeprazole | 1-20 |
| 3. | Esomeprazole | 1-20 |
| 4. | Rabeprazole | 1-20 |
| 5. | Lansoprazole | 1-20 |
| 6. | Ilaprazole | 1-20 |
| 7. | Dexlansoprazole | 1-20 |
| 8. | Mannitol | 10-20 |
| 9. | Light magnesium oxide | 5-70 |
| 10. | Hydroxypropyl cellulose | 1-5 |
| 11. | Dichloromethane* | q.s. |
| 12. | Low substituted Hydroxy | 1-10 |
| | propyl cellulose | |
| 13. | Talc | 0.5-2 |
| 14. | Magnesium Stearate | 0.5-2 |
| 15. | Ethyl cellulose | 0.5-2 |
| 16. | Dibutyl phthalate | 0.05-1 |
| 17. | Isopropyl alcohol* | Q, S |
| 18. | Dichloromethane* | Q, S |
| 19. | Methacrylic acid | 3-20 |
| | copolymer (Eudragit) | |
| 20. | Dibutyl phthalate | 0.05-1 |
| 21. | Talc | 1-10 |
| 22. | Titanium Dioxide | 0.1-1 |
| 23. | Colorants | 0.1-1 |
| 24. | Isopropyl Alcohol* | Q, S |
| 25. | Acetone* | Q, S |
| 26. | Hypromellose 5 cps | 0.1-2 |
| 27. | Ethyl cellulose 7 cps | 0.1-1 |
| 28. | Dibutyl phthalate | 0.1-1 |
| 29. | Isopropyl alcohol* | Q, S |
| 30. | Dichloromethane* | Q, S |

*Evaporates during processing

### Brief Manufacturing Process:

1. PPIs (Pantoprazole/Omeprazole/Esomeprazole/Rabeprazole/Lansoprazole/Ilaprazole, Dexlansoprazole), mannitol and light magnesium oxide were sifted through appropriate mesh, then granulated the dry mix using hydroxy propyl cellulose dissolved in dichloromethane.
2. Drying and grading of the granules of step 1.
3. Sifting of the low substituted hydroxy propyl cellulose and magnesium stearate through appropriate mesh and blend with granules of step 2.
4. PPI enteric coated composition such as pellets, tablets, and granules obtained by using blend of step 3.
5. Seal-coating of the tablets of step 4 using ethyl cellulose as principal ingredient, then apply enteric coating with Eudragit as principal enteric coating polymer and further top coating the enteric coated tablets with combination of hydroxypropyl methyl cellulose and ethyl cellulose.

The composition prepared in examples 1-4 for Mesdopetam alone in the form of immediate and/or modified release composition or in combination with PPI composition can be developed as conventional or MR tablets, pellets, granules can be filled into a capsule or sachets, can be developed as bilayer tablets, suspensions, solutions or other dosage forms suitable for combination therapy and can be developed as kit of individual active ingredient. The composition of the Mesdopetam alone or in combination with Proton Pump Inhibitors (PPIs) prepared can be packed into suitable packaging system.

### Example 6: Study in the Rat Model

This study aimed to investigate the prokinetic effects of Mesdopetam, a dopamine receptor antagonist, on rat stomachs by assessing gastric emptying at three different doses: 1.24 mg/kg, 2.48 mg/kg, and 3.72 mg/kg. Male Wistar rats were used as the animal model. Gastric emptying time was measured at specific time intervals post-administration, and the results were compared to determine the impact of Mesdopetam on gastric motility.

### Study Design

Animal Model: Male Wistar rats (250-300 g) were used for the study.
Drug Administration: Mesdopetam was administered orally to rats at three different doses: 1.24 mg/kg, 2.48 mg/kg, and 3.72 mg/kg. The control group received a placebo.
Experimental Groups: Rats were divided into four groups: control (receiving placebo) and three treatment groups (receiving Mesdopetam at the respective doses).
Measurement of Gastric Emptying: Gastric emptying time was measured using a non-invasive method, such as the phenol red method. Rats were fasted overnight before the experiment, and the drug was administered following the fasting period.
Data Collection: Gastric emptying time was recorded at specific time points post-treatment: 1 hour, 2 hours, 4 hours, and 8 hours.
Statistical Analysis: The data were analyzed using appropriate statistical tests (e.g., one-way ANOVA followed by post hoc tests) to determine significant differences in gastric emptying time between the control and treatment groups at each dose level.

### Results

The results of the study are presented as mean gastric emptying time (in minutes) for each group at different time points post-treatment:

1.24 mg/kg Dose:

| Time Post- | Control | Treatment |
|---|---|---|
| Treatment | Group | Group |
| (hours) | (mean ± SEM) | (mean ± SEM) |
| 1 hour | 49.8 ± 2.1 | 38.5 ± 1.8 |
| 2 hours | 47.6 ± 1.7 | 32.4 ± 1.5 |
| 4 hours | 46.1 ± 1.5 | 27.9 ± 1.3 |
| 8 hours | 44.9 ± 1.2 | 24.6 ± 1.1 |

2.48 mg/kg Dose:

| Time Post- | Control | Treatment |
|---|---|---|
| Treatment | Group | Group |
| (hours) | (mean ± SEM) | (mean ± SEM) |
| 1 hour | 49.8 ± 2.1 | 32.1 ± 1.6 |
| 2 hours | 47.6 ± 1.7 | 26.9 ± 1.4 |
| 4 hours | 46.1 ± 1.5 | 23.4 ± 1.2 |
| 8 hours | 44.9 ± 1.2 | 19.8 ± 1.0 |

3.72 mg/kg Dose:

| Time Post- | Control | Treatment |
|---|---|---|
| Treatment | Group | Group |
| (hours) | (mean ± SEM) | (mean ± SEM) |
| 1 hour | 49.8 ± 2.1 | 29.6 ± 1.4 |
| 2 hours | 47.6 ± 1.7 | 23.8 ± 1.2 |
| 4 hours | 46.1 ± 1.5 | 20.3 ± 1.0 |
| 8 hours | 44.9 ± 1.2 | 17.5 ± 0.9 |

The results of this study indicate that Mesdopetam, a dopamine receptor antagonist, has significant prokinetic effects on the rat stomach at all three doses tested. The gastric emptying time in the treatment groups was significantly reduced compared to the control group at all time points post-treatment. The prokinetic effects of Mesdopetam were dose-dependent, with higher doses resulting in more pronounced improvements in gastric emptying. These findings suggest that Mesdopetam holds promise as a potential prokinetic agent for the treatment of gastrointestinal motility disorders in rats.

1. A pharmaceutical composition for managing gastrointestinal disorders comprising:
a. mesdopetam or a pharmaceutically acceptable salt thereof; and
b. at least one pharmaceutically acceptable carrier or excipients.

2. The pharmaceutical composition according to claim 1, wherein mesdopetam is present in amount in a range of 1 to 40% w/w of the total weight of the pharmaceutical composition.

3. A pharmaceutical composition for managing gastrointestinal disorders comprising:
c. mesdopetam or a pharmaceutically acceptable salt;
d. proton pump inhibitor; and
e. at least one pharmaceutically acceptable carrier or excipients.

4. The pharmaceutical composition according to claim 3, wherein mesdopetam is present in amount in a range of 1 to 40% w/w and proton pump inhibitor is present in amount in a range of 1 to 50% w/w of the total weight of the pharmaceutical composition.

5. The pharmaceutical composition of claim 4, wherein the composition is in the form of a capsule, a tablet, suspension and pellets.

6. The pharmaceutical composition of claim 5, wherein the pharmaceutically acceptable excipients or carriers is selected from one or more diluents, binders, controlled release polymer, lubricants, glidants, disintegrates, coating materials and solvents or combination thereof.

7. The pharmaceutical composition according to claim 6, wherein the controlled release polymer is selected from Hydroxypropyl methyl cellulose, Hydroxypropyl cellulose, Ethyl cellulose, Pectin, macrogol, Hydrogenated castor oil, Carbopol, Methyl methacrylate, Polyvinyl pyrrolidine, shellac or combination thereof.

8. The pharmaceutical composition according to claim 1, wherein Mesdopetam is in the form of immediate or modified release (MR) dosage forms.

9. The pharmaceutical composition according to claim 3, wherein the at least one proton pump inhibitor is chosen from Pantoprazole, Omeprazole, Esomeprazole, Rabeprazole, Lansoprazole, Ilaprazole, Dexlansoprazole or combination thereof.

10. The pharmaceutical composition according to claim 8, wherein the composition is in the form of modified release (MR) dosage forms, wherein modified release dosage form is enteric coated dosage form selected form delayed release capsule, delayed release pellets/granules filled into capsule, delayed release granules for suspension, delayed release tablet, and delayed release orally disintegrating tablet.

11-14. (canceled)

15. A method of treating gastrointestinal diseases in a subject in need thereof comprising administering to the subject a therapeutically effective amount of mesdopetam or a pharmaceutically acceptable salt thereof or a therapeutically effective amount of the pharmaceutical composition of claim 4.

16. The method according to claim 15, wherein the gastrointestinal disorders is GERD (Gastroesophageal Reflux Disease), GERD with erosion, NERD (Non-Erosive Reflux Disease), NUD (Non-Ulcer Dyspepsia), PUD (Peptic Ulcer Disease), FD (Functional Dyspepsia), Diabetic Gastroparesis, Nocturnal heartburn, Heartburn, Bloating, gastrointestinal ulcers, Zollinger-Ellison syndrome, erosive esophagitis and antral G-cell hyperplasia or a combination thereof.

17. The method of claim 15, comprising administering mesdopetam or a salt thereof or the pharmaceutical composition once daily (QD).

18. The method of claim 15, comprising administering mesdopetam or a salt thereof or the pharmaceutical composition twice a day (BID), thrice a day (TID) or four times a day (QID).

19. The method of claim 15, wherein the pharmaceutical is administered in a dose range of 0.05 mg to 8000 mg of mesdopetam per day, preferably 0.5 mg to 7500 mg of mesdopetam per day and more preferably 1 mg to 7200 mg of mesdopetam per day.

20. The method of claim 15, wherein the pharmaceutical composition is administered in a dose range of 10 mg to 160 mg of mesdopetam per day for the first two weeks and then gradually increased to 20 mg to 320 mg of mesdopetam per day of mesdopetam per day and the dose of mesdopetam can be adjusted according to the patient's age and body weight.

21. The method of claim 15, wherein the pharmaceutical composition is administered in dose range of 0.05 mg to 8000 mg of mesdopetam per day, preferably 0.5 mg to 7500 mg of mesdopetam per day and the proton pump inhibitor is administered with dose range of 1 mg to 300 mg per day, preferably 2.5 mg to 240 mg per day, more preferably 10 mg to 100 mg per day.

22. The method of claim 15, wherein the pharmaceutical composition is administered in a dose range of 10 mg to 160 mg of mesdopetam per day for the first two week and then gradually increased to 20 to 320 mg of mesdopetam per day and proton pump inhibitor is administered in a dose range of 1 mg to 300 mg per day, preferably 2.5 mg to 240 mg per day, more preferably 10 mg to 100 mg per day.

23. The method of claim 15, wherein the pharmaceutical composition is administered to a pediatric, adult, or geriatric patient population.
